(19) United States
(12) Patent Application Publication
(10) Pub. No.: US 2020/0093399 A1
(43) Pub. Date: Mar. 26, 2020

(54) BREATH ANALYZER DEVICE

(71) Applicant: Thomas P. Miller, Grand Rapids, MI (US)

(72) Inventor: Thomas P. Miller, Grand Rapids, MI (US)

(21) Appl. No.: 16/619,589

(22) PCT Filed: Jun. 5, 2018
(86) PCT No.: PCT/US2018/035978

§ 371 Date: Dec. 5, 2019

Related U.S. Application Data
(60) Provisional application No. 62/515,764, filed on Jun. 6, 2017.

(51) Int. Cl. A61B 5/083; A61B 5/00; A61B 5/097; A61B 5/087

(52) U.S. Cl. CPC A61B 5/0833; A61B 5/0836; A61B 5/742; A61B 5/0002; A61B 2560/06; A61B 5/0878; A61B 2562/029; A61B 2562/0271; A61B 2560/0431; A61B 5/097

(57) ABSTRACT

A breath analyzer device includes a tube for communicating a breath sample from a user of the breath analyzer device, the tube having a first opening where the breath sample enters and a second opening where the breath sample exits. The tube is adapted to provide minimal restrictions on the flow of the breath sample. The device also includes an oxygen sensor and carbon dioxide sensor disposed partially within the tube and adapted to detecting an amount of oxygen and carbon dioxide present in the breath sample respectively. The device also includes a controller adapted to receive data from the oxygen sensor and the carbon dioxide sensor.

### FIELD OF THE INVENTION

The present invention relates generally to devices used to analyze lung function, such as a spirometer or the like, and are operable to determine diagnosis and treatment decisions.

### BACKGROUND OF THE INVENTION

Hyperpolarized gas and xenon magnetic resonance imaging (MRI) are known technologies that can give information regarding lung microstructure and regional function or ventilation. These technologies are expensive and are not available for point of care testing. Further, current methods offer a tradeoff between sensitivity to small airways and ease of use.

### SUMMARY OF THE INVENTION

The present invention provides a breath analyzer device that allows for unrestricted breathing by a user of the device during testing. More particularly, the device allows for evaluation of the small airway function of lungs, by monitoring air constituents, without restricting the user's breath.

In some implementations, a breath analyzer device includes a body for communicating a breath sample from a user of the breath analyzer device. An oxygen sensor is adapted to detecting an amount of oxygen present in the breath sample and a carbon dioxide sensor is adapted to detecting an amount of carbon dioxide present in the breath sample. A controller is adapted to receiving data from the oxygen sensor and the carbon dioxide sensor.

In other implementations, a breath analyzer device includes a tube for communicating a breath sample. The tube has a first opening where the breath sample enters and a second opening where the breath sample exits. The tube is adapted to provide minimal restrictions on a flow of the breath sample. The device also includes an oxygen (O₂) sensor disposed partially within the tube and adapted to detecting an amount of oxygen present in the breath sample. A carbon dioxide (CO₂) sensor is also disposed partially within the tube and adapted to detecting an amount of carbon dioxide present in the breath sample. A controller is included and adapted to receive data from the oxygen sensor and the carbon dioxide sensor.

Optionally, the breath analyzer device includes a display adapted to receive data from the controller and provide the received data visually. The breath analyzer device may include a wireless interface module adapted to receive data from the controller and wirelessly transmit the data to a remote receiver. The breath analyzer device may include memory adapted to store data received from the controller. A mouthpiece may be in fluid communication with the first opening of the tube, and the breath sample enters the mouthpiece prior to entering the first opening of the tube. The mouthpiece optionally includes a surface coated with a bacteriostatic agent or filter to restrict bacteria, viruses or other contaminants from entering the tube. The breath analyzer device, in some examples, includes a humidity sensor adapted to detecting an amount of water vapor present in the breath sample. The controller is then adapted to receive data from the humidity sensor. Optionally, the breath analyzer device also includes a temperature sensor adapted to detecting a temperature of the breath sample, and the processor is adapted to receive data from the temperature sensor. The breath analyzer device, in some implementations, includes a handle coupled to the tube for a user to grasp while using the breath analyzer device. The breath analyzer device may include a wired interface module adapted to receive data from the controller and transmit the received data through a wired interface to a remote receiver. The breath analyzer device may wirelessly transmit received data to a remote receiver.

In another implementation, a breath analyzer device includes a pre-valve subsystem for accepting a breath sample and communicating the breath sample. The device also includes at least one check valve for receiving the communicated breath sample and isolating the breath sample from other gases, where the at least one check valve is adapted to add minimal differential pressure to the breath analyzer device. A carbon dioxide sensor is adapted to detecting an amount of carbon dioxide in the breath sample, and an oxygen sensor is adapted to detecting an amount of oxygen in the breath sample. The device also includes a post-valve subsystem for receiving the communicated breath sample and expelling the breath sample from the breath analyzer device and a controller adapted to receive data from the oxygen sensor and the carbon dioxide sensor.

Optionally, the breath analyzer device includes a heater adapted to heat at least one of the carbon dioxide sensor and oxygen sensor. The heater may include heating tape. The pre-valve subsystem may have a total dead space of less than 185 ml. In some examples, the post-valve subsystem includes a post-valve tubing extension adapted to minimize diffusive flow of the breath sample. The pre-valve subsystem may include a disposable mouthpiece. In some implementations, the oxygen sensor includes a Teflon membrane. The post-valve subsystem may be manufactured of polyvinyl chloride (PVC). The breath analyzer device, in some examples, is adapted to provide a differential pressure lower than 10% of positive-end expiratory pressure.

These and other objects, advantages, purposes and features of the present invention will become apparent upon review of the following specification in conjunction with the drawings.

### BRIEF DESCRIPTION OF THE DRAWINGS

FIG. 1 is a rear perspective view of a breath analyzer device in accordance with the present invention;

FIG. 2 is an exploded rear perspective view of the breath analyzer device of FIG. 1;

FIG. 3 is a front perspective view of the breath analyzer device of FIG. 1;

FIG. 4 is a side plan of the breath analyzer device of FIG. 1;

FIG. 5 is a top plan view of the breath analyzer device of FIG. 1;

FIG. 6 is a rear plan view of the breath analyzer device of FIG. 1;

FIG. 7 is a perspective view of another breath analyzer device;

FIG. 8 is a perspective view of the breath analyzer device of FIG. 7;

FIG. 9 is a perspective view of a pre-valve subsystem of the breath analyzer device of FIG. 7;

FIG. 10 is a perspective view of a mouthpiece of the breath analyzer device of FIG. 7;

FIGS. 11A and 11B are perspective views of a first pre-valve tubing part and a second pre-valve tubing part of the pre-valve subsystem of FIG. 9;

FIG. 12 is a perspective view of check valves of the breath analyzer device of FIG. 7;

FIG. 13 is a schematic of a CO₂ sensor;

FIG. 14 is a plot of data acquired from a CO₂ sensor;

FIG. 15 is a graph of data acquired from a CO₂ sensor;

FIG. 16 is a schematic of an O₂ sensor;

FIG. 17 is a plan view of processing device in accordance with the present invention;

FIG. 18 is a graph of data acquired from an O₂ sensor;

FIG. 19 is a perspective view of a post-valve subsystem of the breath analyzer device of FIG. 7;

FIG. 20 is a perspective view of post-valve tubing of the post-valve subsystem of FIG. 19;

FIG. 21 is a perspective view of a CO₂ sensor attachment piece;

FIG. 22 is a perspective view of an O₂ sensor attachment piece; and

FIG. 23 is a schematic of a post-valve tubing extension in accordance with the present invention.

### DESCRIPTION OF THE PREFERRED EMBODIMENTS

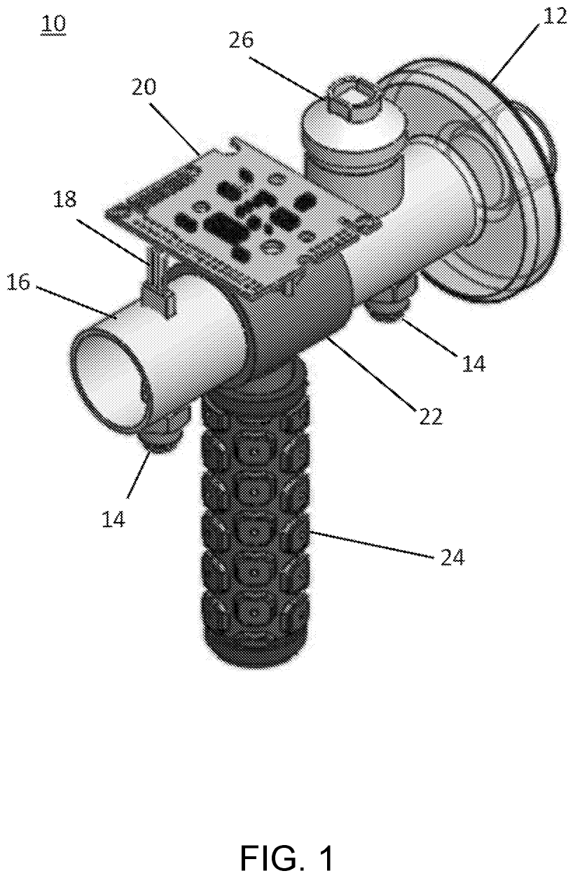

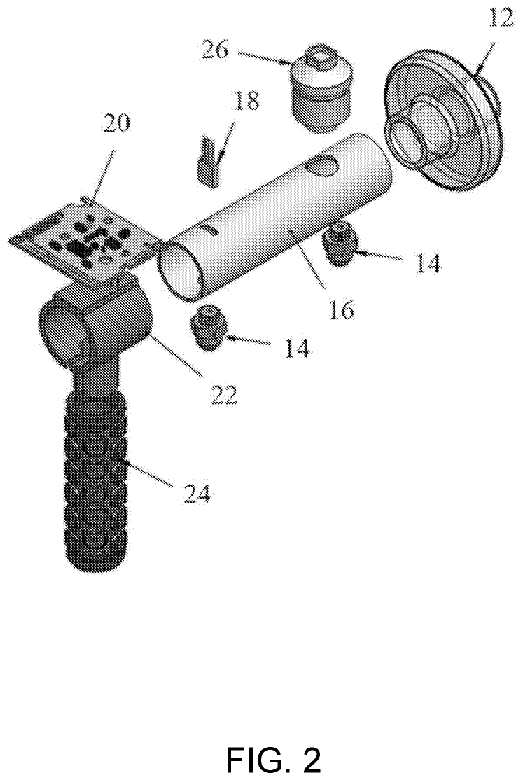

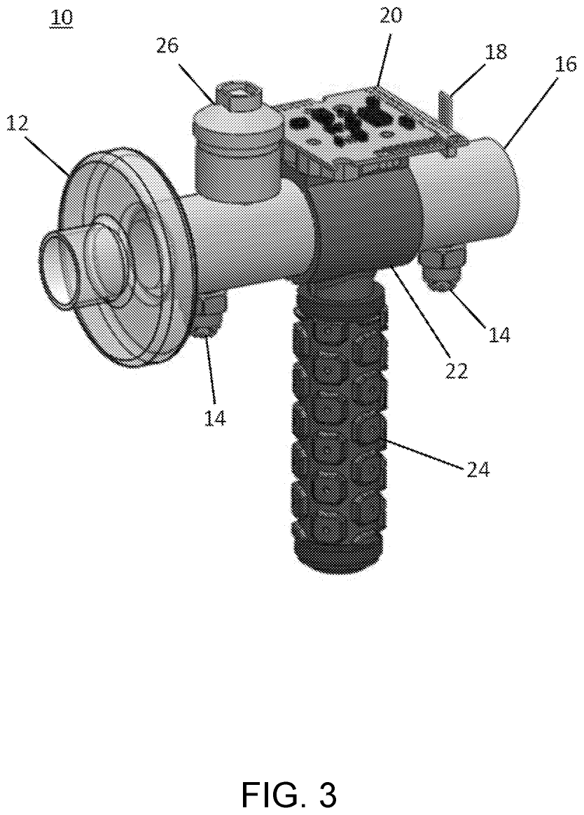

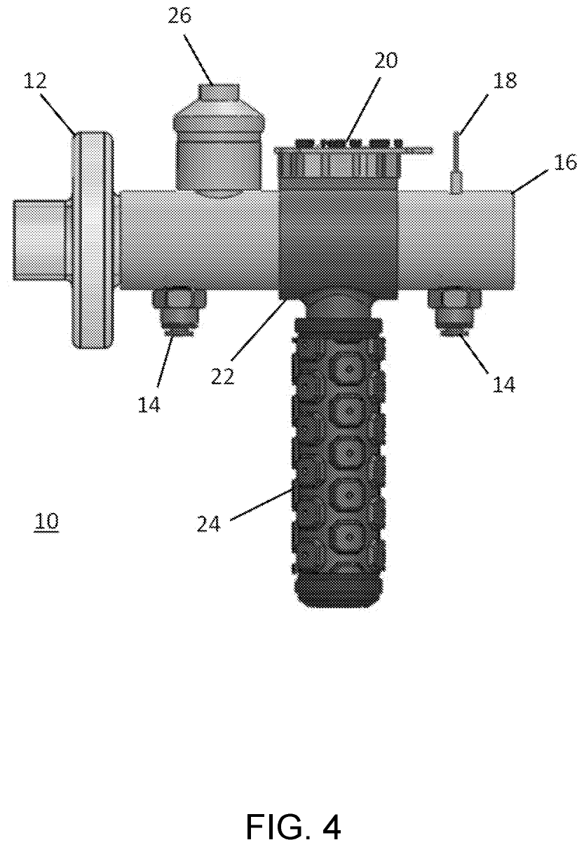

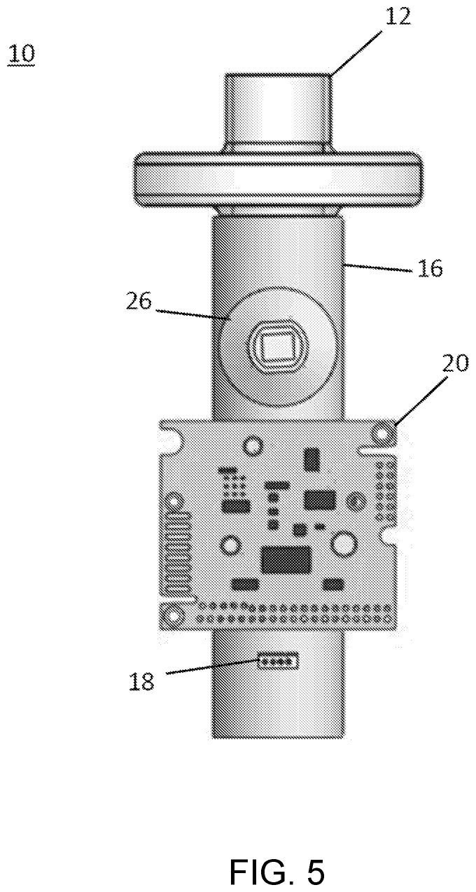

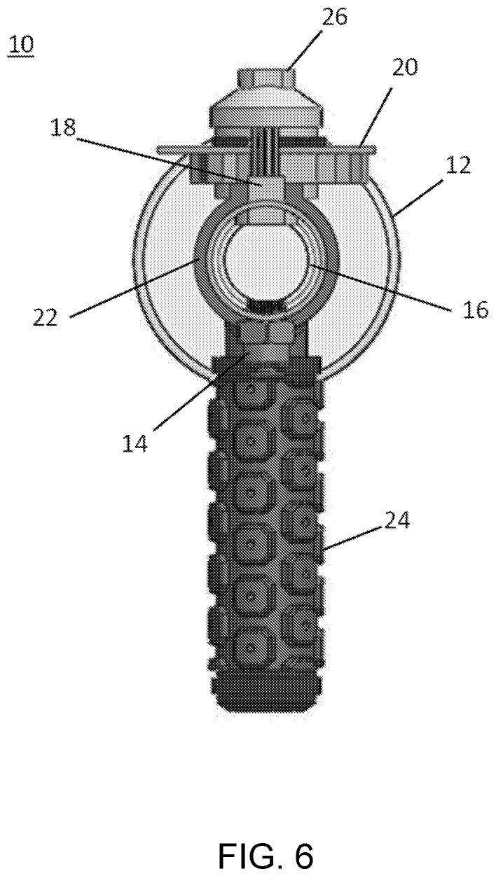

Referring now to the drawings and the illustrative embodiments depicted therein, a breath analyzer device 10 is configured to receive a breath sample (FIGS. 1-6). The breath analyzer device 10 includes a tube 16 having a first end where breath sample enters and a second end where the breath sample exits. The tube 16 is configured to not restrict the flow of the user's breath. The breath analyzer device 10 includes an oxygen sensor 26 that is disposed partially within the tube 16. The breath analyzer device 10 also includes a carbon dioxide sensor 18 that is disposed partially within the tube 16. The oxygen sensor 26 and carbon dioxide sensor 18 are used to determine the levels of oxygen and carbon dioxide in the user's breath during use of the device, as discussed below.

Conventional lung function testing, such as spirometry and plethysmography, are widely available and easy to perform. However, these tests lack sensitivity to the small airways, thus preventing them from being effective in early detection of lung diseases such as chronic obstructive pulmonary disease (COPD). For example, spirometry is one of the most common pulmonary function tests. However, a spirometer only reflects the user's ability to inhale and exhale; i.e., generate air flow. While useful, spirometry lacks information about the ventilatory functioning of the small airways of the lungs or the lungs' ability to perform the important function of gas exchange. Other methods, such as inert gas washout, are highly sensitive to the small airways but difficult to perform and are very time consuming. Therefore, there is an evident need in the market of lung disease detection methods for an effective technique that can detect early stage disease in clinical settings.

Ideally, the technique will offer a combination of minimal air mixing, low differential pressure across the device (i.e., a low change in pressure between the inlet pressure (exhale pressure by the user) and the outlet pressure at the outlet of the device), functionality in the presence of high humidity, and reusability. In the present invention, the volume of the initial ventilation portion of the device, including the mouthpiece, check valves, and bifurcating tube, is of the appropriate size to prevent excessive air mixing within the device. The mixing of inhaled and exhaled air can alter the concentration readings of the CO₂ and O₂ sensors, following the initial portion of the device. To minimize this effect, the dead space volume of the device is carefully considered, as discussed in detail below. Low differential pressure across the device improves the accuracy of disease detection. Further, because expired air is nearly fully saturated with water vapor (H₂O), the CO₂ and O₂ sensors of the device remain functional in the presence of high humidity. The device is reusable because the user may easily clean or dispose of the ventilation portion of the device. The mouthpiece may be disposed of and replaced after each use, and the check valves and bifurcating tube may be cleaned with alcohol.

By not restricting the user's breath, the device 10 allows the user's breath to be sampled at a baseline level without a change in breathing patterns necessary. It is crucial to minimize resistance during respiration to ensure that the status of the small airways are similar to their baseline state. Such change in breathing patterns or breathing against resistance, can alter or mask the results of the true functioning of the lungs e.g., exhaling against resistance will artificially keep small airways open, thus changing their capacity for air exchange and thus altering the results observed compared to a baseline or natural state. After a baseline is established, respiratory maneuvers, such as during exercise or forced vital capacity maneuvers, may be conducted to gain additional information. In addition, the present invention could be combined with a standard spirometer in order to determine both the ventilation and gas exchange characteristics, in addition to standard spirometric parameters, with a single integrated device.

During use of the device, a user can hold a handle portion 24 and breathe in and out through the intake end 12. The oxygen sensor 26 is adapted to detect an amount of oxygen in the breath sample passing through the tube 16. The oxygen sensor 26 communicates the data gathered from the breath sample to a controller 20. The illustrated embodiment depicts the controller 20 mounted to the top of the tube 16, but it may be mounted in any fashion that does not impede the use of the tube 16.

The carbon dioxide sensor 18 is adapted to detecting an amount of carbon dioxide in the breath sample passing through the tube 16. The carbon dioxide sensor 18 also communicates the data gathered from breath sample to the controller 20. Normal breathable air is approximately 21% oxygen and 0% carbon dioxide. Typically, exhaled air from a human is approximately 16% oxygen and 4% carbon dioxide. This difference reflects the normal air exchange that occurs during ventilation. Disruption of this air exchange may result in changes (increases and/or decreases) to these typical numbers. For example, the oxygen percentage could be higher than 16% or the carbon dioxide percentage could be below 4%. The percentages may also be affected by respiratory maneuvers such as hyperventilation or hypoventilation or dynamic hyperinflation, or applying the test under various conditions such as exercise. In the illustrated embodiment, the oxygen sensor 26 and carbon dioxide sensor 18 will detect these concentrations and communicate the data to the controller 20.

The breath analyzer device 10 may also include a humidity sensor adapted to detect an amount of water vapor present in the user's breath sample. The humidity sensor may also communicate the data gathered from the breath sample to the controller 20. The breath analyzer device 10 may also include a temperature sensor adapted to detecting a temperature of the breath sample. The temperature sensor may also communicate the data gathered from the breath sample to the controller 20. By detecting the humidity and temperature of the breath sample, the controller 20 may alter or correct analysis of the oxygen and carbon dioxide data received from the other sensors. This allows for a more accurate representation of lung function. As shown in the illustrated embodiment, the breath analyzer device 10 may also include at least one pressure fitting sensor 14. A pressure fitting sensor 14 may be partially disposed in the tube 16 at both the first end and the second end. The pressure fitting sensors 14 detect the pressure differential between the interior of the tube 16 and the exterior of the tube 16 where the sensor 14 is disposed.

As shown in the illustrated embodiment, the breath analyzer device 10 may include a handle portion 24. The handle provides a convenient grasping location for the user or for the attending doctor, nurse, or assistant without impeding the use of the tube 16. As illustrated, the handle may be coupled to a mount 22, wherein the mount 22 encircles the tube 16 and securely holds the handle portion 24 in a position approximately orthogonal to the tube 16. As also shown in the illustrated embodiment, the breath analyzer device 10 may also include a mouthpiece 12 at the intake end that is at least partially comprised of a surface coated with a bacteriostatic filter or agent (used to prevent the growth of bacteria on surfaces). The mouthpiece 12 may be coupled to and in fluid communication with the first opening or intake of the tube 16. The mouthpiece 12 is configured such that a breath sample first enters the mouthpiece 12 and then enters the first end of the tube 16. The mouthpiece may also be disposable or include a filter.

The breath analyzer device 10 may also include a display that is adapted to receive data from the controller 20 and then provide the received data visually. Such a display could be used to immediately communicate results of the data gathered from the sensors. The display could take any form suitable for communicating the information visually. For example, the display could be a liquid crystal display or a series of light emitting diodes. The breath analyzer device 10 may also include a wireless interface module that is adapted to receive data from the controller 20 and wirelessly transmit the data to a remote receiver. The wireless interface module may implement any number of wireless technologies appropriate for communicating data. For example, the wireless interface module may implement Wi-Fi or Bluetooth. The remote receiver may be any device appropriate for receiving the data, such as a mobile phone, tablet, laptop, or personal computer. The breath analyzer device 10 may also use a wired interface module to connect with a remote receiver. Such an interface could comprise of any number of appropriate protocols, such as Ethernet or USB.

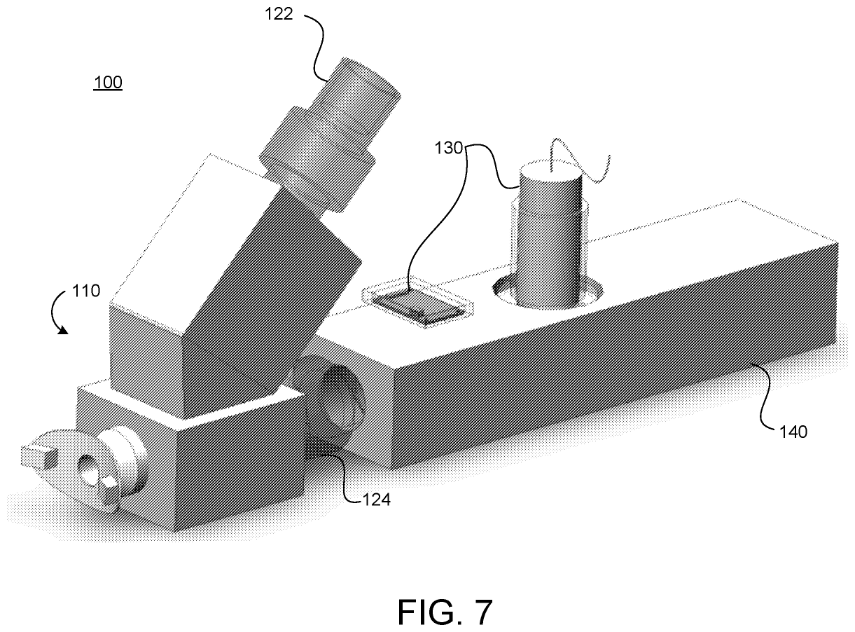

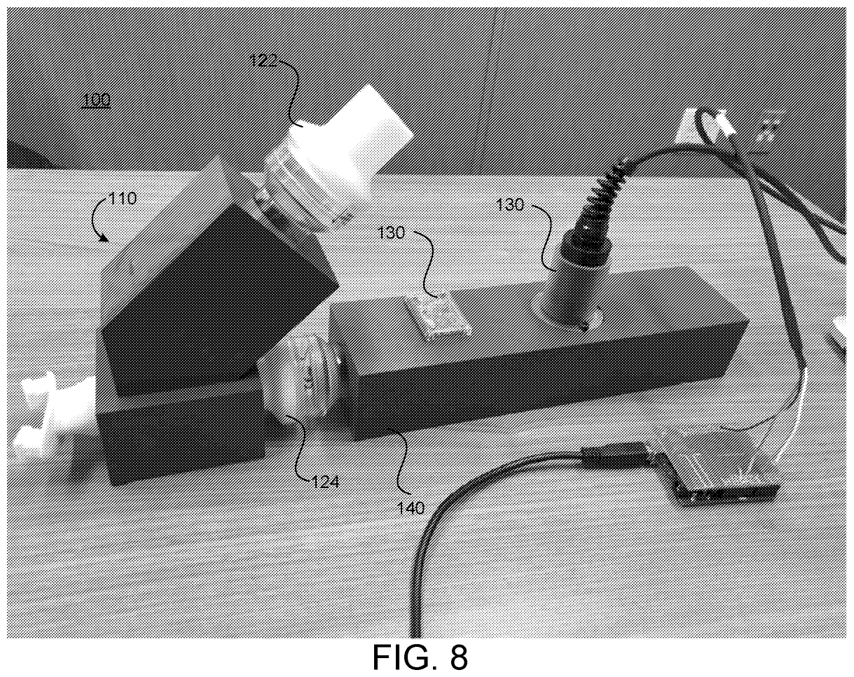

In other implementations, as shown in FIGS. 7 and 8, the device 100 is characterized by four distinct sections: a pre-valve subsystem 110 (e.g., mouthpiece and pre-valve tubing), valves 120 (such as unidirectional check valves), CO₂ and O₂ sensors 130, and a post-valve subsystem 140 (e.g., post-valve tubing and sensor attachment pieces). The pre-valve subsystem 110 and check valves 120 direct the flow and ensure the isolation of inspired and expired air. Following the exhalation valve are the CO₂ and O₂ sensors used to characterize exhaled flow. The remaining portion of the device 100 is an extended tube for exhaled air to flow out of after analysis.

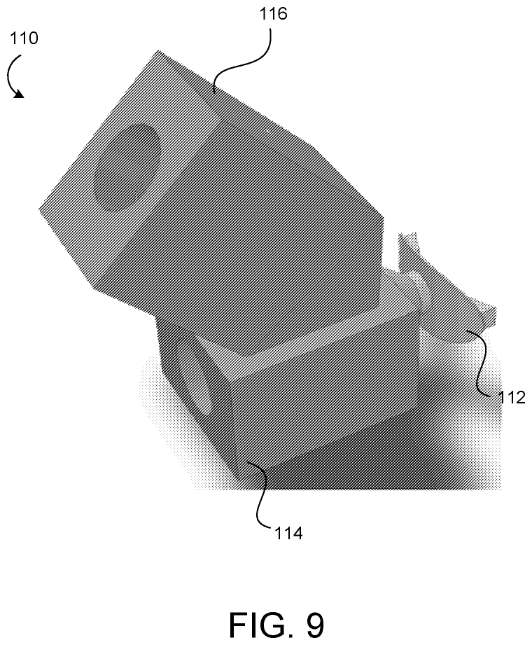

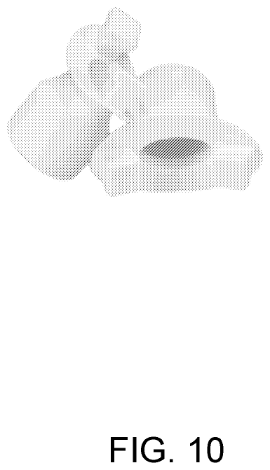

Referring now to FIG. 9, the pre-valve subsystem 110 includes a mouthpiece 112 and a first pre-valve tubing part 114 and a second pre-valve tubing part 116 connected via dowel pins (not shown). Ideally, the mouthpiece 112 is disposable. The mouthpiece 112 may be, as shown in FIG. 10, a disposable thermoplastic mouthpiece from A-M SYSTEMS™. The mouthpiece 112 consists of an area for the user to put inside their mouth, with teeth blocks for comfort, and a tubular area that will connect the mouthpiece 112 to the rest of the device 100. The entire mouthpiece 112 is ideally made of medical grade material (e.g., Santoprene™ thermoplastic rubber), which allows for flexible connection to the first pre-valve tubing part 114. The mouthpiece 112 specifications shown in Table 1 are illustrative only, and a variety of dimensions may be used while still maintaining a tight, leak-proof connection with the device 100.

TABLE 1
Exemplary Mouthpiece Specifications

| | | |
|---|---|---|
| Inner Diameter | 1.13 | inches |
| Outer Diameter | 1.35 | inches |
| Total Volume, Unconnected | 30 | ml |
| Connector Length | 0.82 | inches |
| Total Length | 1.54 | inches |

While a disposable mouthpiece 112 is illustrated, a reusable mouthpiece may be substituted. However, because reusable mouthpieces are typically constructed of autoclavable materials, reusable mouthpieces have larger dead space than a comparable disposable mouthpiece. Dead space, or total volume, of the mouthpiece 112 should be as small as possible to ensure that excess ambient air inhaled through the check valves, but that does not participate in gas exchange within the lungs, will not affect O₂ and CO₂ concentration measurements by more than 0.5 percent.

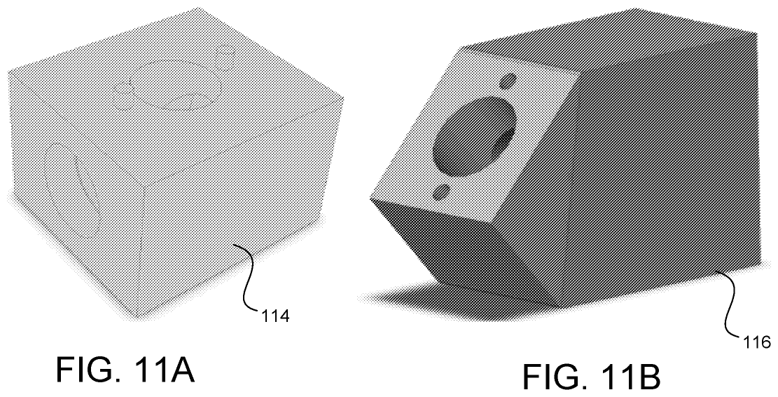

FIGS. 11A and 11B illustrate the first pre-valve tubing part 114 (FIG. 11A) and a second pre-valve tubing part 116 (FIG. 11B). The pre-valve tubing 114, 116 may, for example, have an inner diameter of 1.25 inches through which air will flow. In the first pre-valve tubing part 114, one side is fitted to a diameter of, for example, 1.32 inches to create a snug fit for the mouthpiece 112. The other side may be fitted to a diameter of 1.35 inches to create a snug fit for the unidirectional exhalation check valve. The angled side of the second pre-valve tubing part 116 may be fitted to a diameter of 1.35 inches to also create a snug fit for the unidirectional inhalation check valve. Ideally, the pre-valve tubing minimizes total volume. For example, the total volume of the pre-valve tubing 114, 116 may be 150 ml to 200 ml. The total volume of the pre-valve tubing 114, 116 may be 170 ml.

The volume within the pre-valve subsystem represents a dead space, or volume, in which gas exchange does not occur, but is still mixed with the exhaled air upon expiration and therefore contributes to measurements of CO₂ and O₂. Since inhaled air and exhaled air have significantly different concentrations, any added ambient air due to this dead space will result in exhaled CO₂ lower than expected and exhaled O₂ higher than expected. However, this trend in differences between CO₂ and O₂ concentrations is the exact direction of change expected for patients with lung disease (such as COPD) compared to healthy individuals. Therefore, if too large, the dead space between the valves and the user's mouth could lower the differences in concentrations between a healthy individual and one with lung disease enough that differences are indistinguishable.

To combat this, the maximum allowable dead space volume (VD) is determined by analyzing the air mixing of inhaled and exhaled concentrations of CO₂ and O₂ in healthy individuals. With an estimated tidal volume during submaximal exercise of 1000 ml, VD is calculated using the following equation:

$${V}_{D} = \frac{{V}_{tidal} ( {C}_{final} - {C}_{exhaled} )}{{C}_{inhaled} - {C}_{final}}$$

Using typical expected CO₂ and O₂ concentrations of healthy individuals, this equation determines a maximum allowable dead space volume, V_{D}, of 113.6 ml and 144.1 ml for O₂ and 002, respectively. Ideally, the dead space volume of the device 100 is near or below these values. For example, the total volume of the device may be less than 200 ml, or, ideally, less than 185 ml, which will not significantly reduce the expected difference between healthy users and those with lung disease. With a dead space area of 185 ml, it is expected that in healthy individuals exhaled O₂ and CO₂ will be approximately 16.8% and 3.4%, respectively. There is also an anatomical dead space within users' lungs that is noteworthy when considering the effect of dead space on sensor readings. Dead space volume within the lungs averages 150 ml, and includes the volume in which no gas exchange occurs. However, since this dead space is present for all individuals, healthy or with obstructive lung disease, it is already accounted for in the standard inhaled and exhaled air concentrations. Therefore, anatomical dead space is typically not a concern for measurements by the device 100 because the objective is to look for deviations in exhaled concentration from those already accepted values.

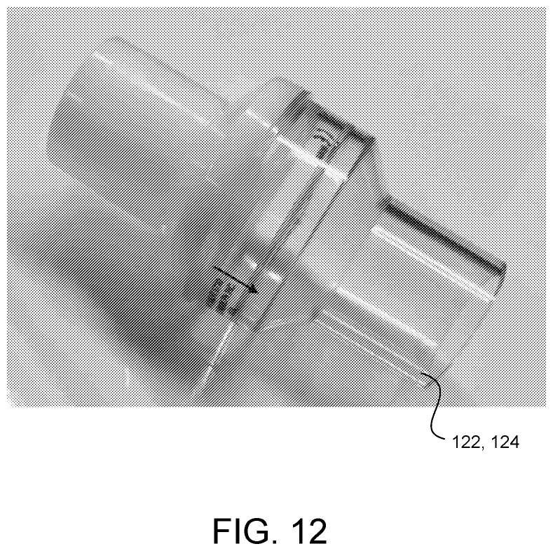

Referring now to FIG. 12, check valves 122, 124 are used to ensure the isolation of inhaled and exhaled air as the user breathes in and out of the mouthpiece 112. Check valves function by only permitting gas flow in one direction in response to pressure. When the user inhales through the mouthpiece 112 of the device 100, inspiratory pressure will force the upper check valve 122 to open, allowing ambient air to be inhaled. After inhalation, that same check valve 122 will shut close, disallowing more ambient air from entering the system. Upon exhalation, expiratory pressure will force the lower check valve 124 open, allowing the exhaled air to pass through the rest of the device 100. The lower check valve 124 will then close again after the pressure of exhalation ceases. Ideally, the check valves will add minimal differential pressure. For example, Harvard Apparatus 60-3174 check values may be used. The check valves 122, 124 specifications shown in Table 2 are illustrative only, and a variety of dimensions may be used while still providing a low differential pressure.

TABLE 2
Exemplary Check Valve Specifications

| | | |
|---|---|---|
| Inner Diameter | 1.126 | inches |
| Outer Diameter | 1.375 | inches |
| Length of Inhalation Port | 1.2 | inches |
| Length of Exhalation Port | 1.2 | inches |
| Differential Pressure at a flow of 100 L/min | 0.5 | cm H₂O |

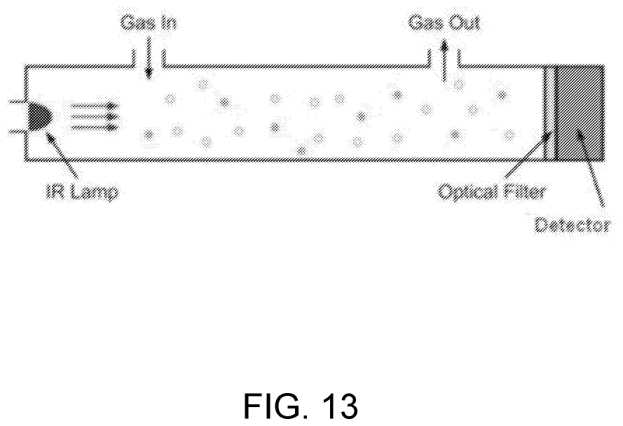

The most common CO₂ sensors utilize Non-Dispersive Infrared (NDIR) technology, commonly used in capnography. NDIR sensors utilize CO₂'s characteristic absorption to detect its concentration. Key components are an infrared (IR) source, a light tube, an interference wavelength filter, and an infrared detector. Referring now to FIG. 13, an IR light is projected through a gas, passed through an optical filter to focus on the desired gas component, and read by an intensity detector. CO₂ is known to have high absorbance in the infrared region of the electromagnetic spectrum at wavelengths of 2.7, 4.3, and 15 μm. The wavelength of 4.3 μm has been demonstrated to have maximum absorption and minimal interference for CO₂, so this waveband is generally used in the detectors. The sensor then uses Lambert-Beer's equation to determine the concentration of the gas and outputs the resulting intensity difference as a voltage to a computing device. The sensor's size, accuracy, and speed of measurement are all important considerations. The sensor, for example, may be a SprintiR Fast 20% CO₂ sensor. The CO₂ sensor may include heating (e.g., via heating tape) to ensure condensation (from humidity) does not form on the sensor.

TABLE 3
Exemplary CO₂ Sensor Specifications

| | |
|---|---|
| Sensing Method | NDIR absorption, Gold-plated optics, Solid-state |
| Sample Method | Diffusion (Standard)/Flow through (with flow- |
| | through adapter) |
| Measurement Range | 0-20% |
| Accuracy | ±70 ppm +/− 5% of reading |
| | (100% Range ± 300 ppm +/− 5% of reading) |
| Measurement Noise | <10% of reading with no digital filtering |
| Non Linearity | <1% of FS |
| Pressure | 0.1% of reading per mbar in normal atmospheric |
| Dependence | conditions |
| Operating Pressure | 950 mbar to 10 bar |
| Range | |

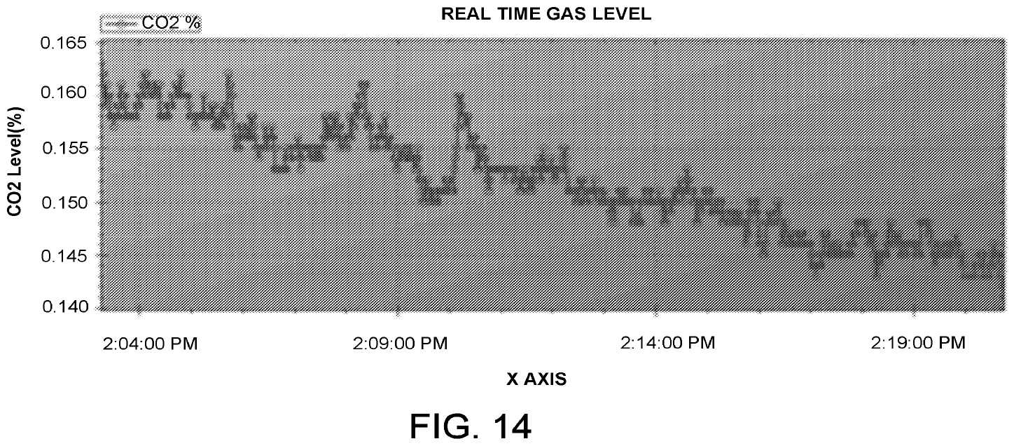

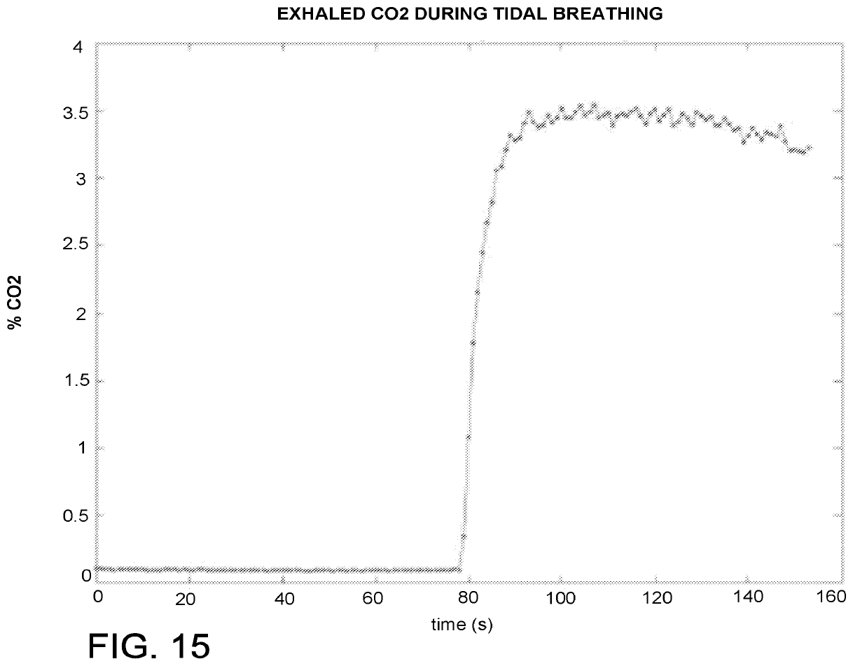

Sensor software, executed by processing hardware, may interface with the CO₂ sensor (directly or indirectly) to manage the sensor's data. As shown in FIG. 14, a graph of acquired data given a specified sampling rate and chosen output concentration (PPM or percent) may be generated by the sensor software. The sensor software may allow for configuration and calibration of the sensor. The sensor, for example, may be calibrated by being exposed to open airs (e.g., outdoors) and then confirmed by testing with known concentrations of CO₂. The sensor software may include the ability to analyze the collected data, or, alternatively, the data may be exported to data analysis software (e.g., MATLAB). Referring now to FIG. 15, a plot of CO₂ percentage versus time for exhaled CO₂ during tidal breathing is illustrated. Optionally, the device 100 includes a warmer to warm the CO₂ sensor in order to prevent condensation forming on the sensor that may lead to inaccurate readings.

Optionally, the device 100 includes a flow sensor to determine if a state of laminar, transient, or turbulent flow is affecting the concentration readings of O₂ and CO₂ detected by the sensors.

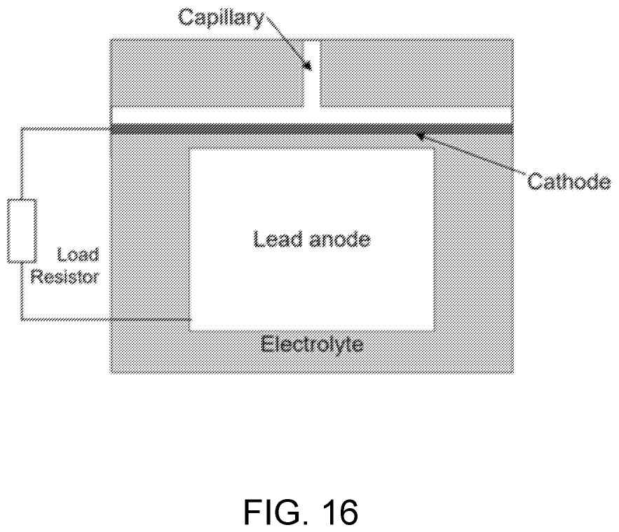

The most common technology used for measuring O₂ concentration in air is a Galvanic mechanism. Galvanic sensors operate like a metal/air battery. O₂ that comes in contact with the cathode, usually composed of gold, is reduced to hydroxyl ions, and a balancing reaction of lead oxidation takes place at the anode. These sensors generate a current proportional to the rate of O₂ consumption (following Faraday's law). As shown in FIG. 16, the current is measured using a load resistor between the anode and cathode and measuring the voltage drop. The load resistor is typically between 10 and 100 Ohms because a low resistance results in a small voltage drop difficult to measure, and a high resistance imposes a voltage across the anode and cathode that can cause side reactions. This mass flow control O₂ sensor shows a weak temperature dependence, due to the change of gas viscosity with temperature, and almost no pressure dependence because reduction of the cathode depends on concentration, not partial pressure.

The lifetime of the O₂ sensor is dependent on the availability of lead, as the sensor becomes unusable when all of the lead has oxidized. The most common electrolyte in these sensors is potassium hydroxide (KOH) because lead oxidation is best controlled in an electrolyte with a pH between 10 and 12. Lead oxide also occupies more volume than pure lead, so the combined anode and electrolyte volume inside the O₂ sensor increases with use. If the sensor is not manufactured properly, the internal pressure inside will end up splitting the sensor case, resulting in leakage.

Alternatively, O₂ in gas may be measured using a solid Teflon-like membrane. The rate of gas diffusion through this membrane is linearly proportional to the partial pressure of O₂ on both sides of the membrane, according to Fick's Law. As O₂ is being reduced at the cathode, the partial pressure on the cathodic side of the membrane is close to zero, proving that there is a driving force linearly dependent on oxygen partial pressure leading to an output that is also linearly dependent on this pressure. This method also uses temperature compensation due to temperature dependence of the solid polymer membrane. Such sensors tend to have a slower response time than the capillary method, because of the slower diffusion through a membrane than a capillary. However, there is less variation in the data when exposed to pressure changes. The O₂ sensor, for example, may be a Fast Response Thermistor Reference Oxygen Sensor of Apogee Instruments Inc. Such a device does not require a thermocouple reference or amplification because the primary focus is not the temperature of exhaled air. The sensor may be heated (e.g., with heating tape) to prevent condensation from forming on the sensor.

TABLE 4
Exemplary O₂ Sensor Specifications

| | |
|---|---|
| Diameter | 3.15 cm |
| Length | 6.85 cm |
| Range | 0 to 100% O₂ |
| Accuracy | <0.01% O₂ drift per day |
| Repeatability | ∓0.001% O2 (10 ppm) |
| Input Power | 12 V power for heater, 5 V |
| | excitation for thermistor |
| Operating Environment | 0 to 50° C. |

The sensor may require calibration before normal operation. For example, the sensor may be calibrated by using two measurement points. Because the sensor provides a linear relationship between the voltage output and the O₂ concentration in the air, calibration consists merely of a calibration factor, which is a multiplier to be applied to all measurements. Using the two measurement points, the calibration factor is determined.

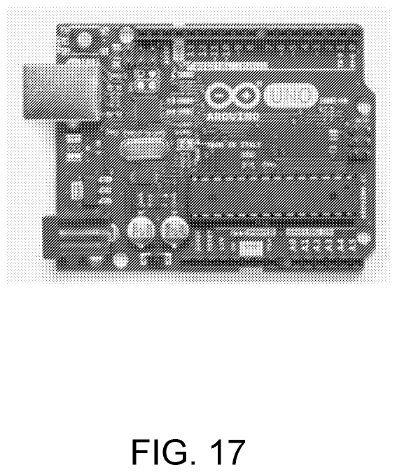

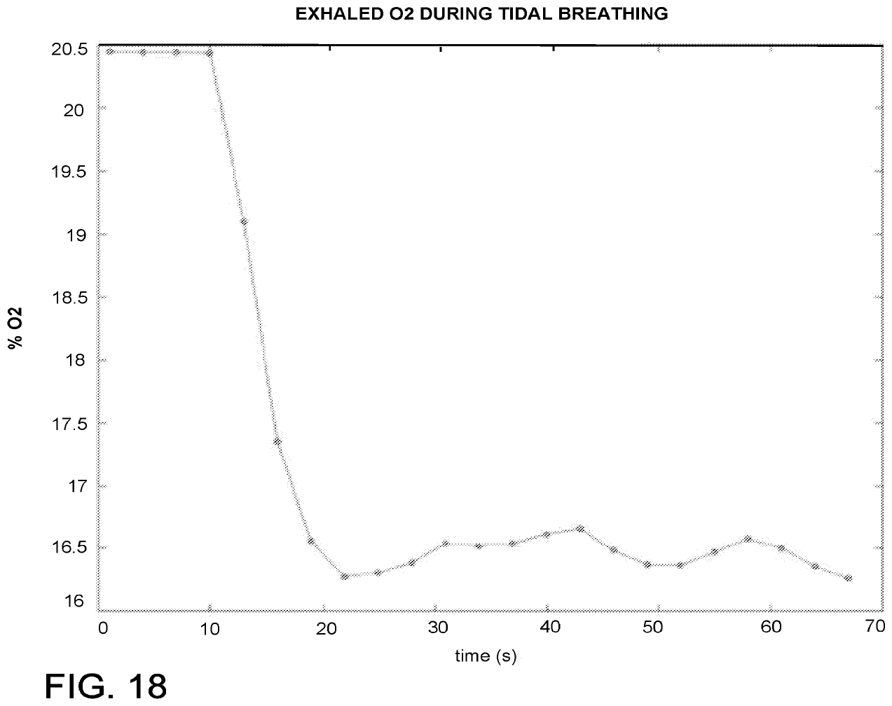

The O₂ sensor may be connected to processing hardware, for example, an Arduino Uno. FIG. 17 illustrates such processing hardware. The data may be processed with data analysis software (e.g., MATLAB). FIG. 18 illustrates a plot of data captured by the O₂ sensor. The data represents exhaled O₂ percentage during tidal breathing as a function of time.

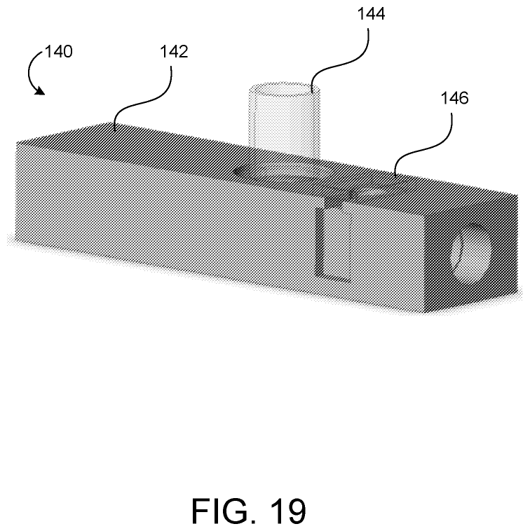

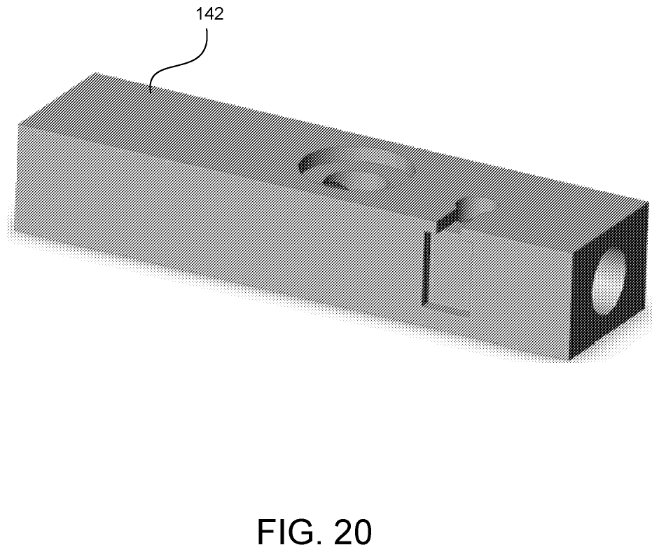

Referring now to FIG. 19, the post-valve subsystem 140 includes the post-valve PVC tubing 142 as well as attachment pieces 144, 146 for the O₂ and CO₂ sensors respectively. The post-valve tubing 142 guides exhaled air through the exhalation check valve, past the CO₂ and O₂ sensors, respectively, and into the environment. FIG. 20 illustrates the post-valve tubing 142 alone. The post-valve subsystem 140 includes a rectangular PVC bar through which a circular hole is bored. For example, the hole may be 1.25 inches in diameter. The diameter is ideally smaller than the outer diameter of the check valves so that a snug fit is maintained. Referring back to FIG. 20, two holes may be drilled on the top face of the post-valve tubing for the sensors to attach (although the sensors may be attached elsewhere). In addition, additional material may be removed from the side of the PVC to allow for wiring to connect to the CO₂ sensor. A circular mounting location facilitates the mounting of the O₂ sensor attachment piece 144. PVC is an ideal material because it is durable enough to withstand the downward force of the two sensors attached and can be bored easily without the risk of stress fractures. Additionally, PVC is a chemically stable material. PVC is nonoxidizing and does not undergo significant changes in composition or properties over time. Because the tubing will mostly come into contact with common gases in ambient and exhaled air, this is an added benefit that ensures that the properties of the tubing itself will not influence sensor readings. However, other suitable materials may be used. A rectangular PVC bar instead of a circular bar allows the outer walls of the tubing will be flat. This is advantageous because the flat sides facilitate the attachment of the attachment pieces that will encapsulate the two sensors.

During tidal breathing, the airflow through the post-valve tube will remain laminar. However, during elevated breathing (e.g., during exercise), the larger flow rates in exhalation may cause the flow to reach a transient level. However, due to the diameter of the check valves and length of the device 100, such transient flow will not affect sensor readings.

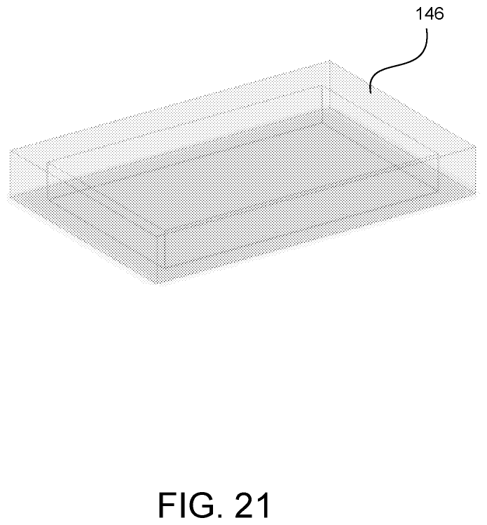

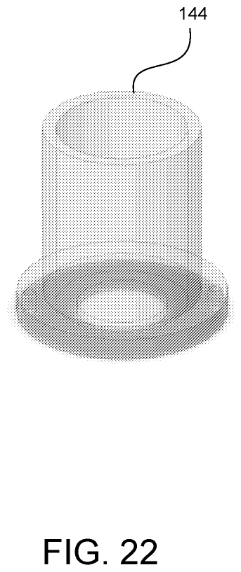

FIG. 21 illustrates a CO₂ sensor attachment piece 146. The CO₂ sensor attachment 146 piece protects the CO₂ sensor and secures it in place. The CO₂ sensor attachment 146 may be constructed of any suitable material, but acrylic, because of its transparent properties, facilitates wiring and troubleshooting. The CO₂ sensor attachment may connect to the post-valve tubing via screws to allow for easy detachment. Referring now to FIG. 22, an O₂ sensor attachment piece 144 protects the O₂ sensor and also secures the O₂ sensor in place by creating a snug fit with the O₂ sensor. The O₂ sensor attachment piece 144 may also be constructed of acrylic and mounted to the post-valve tubing via screws.

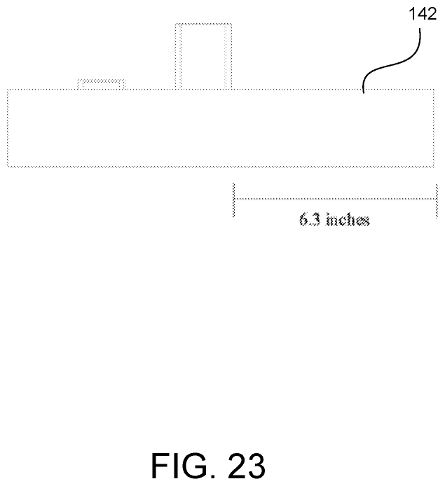

Referring now to FIG. 23, after the O₂ sensor intersection, an additional length of tubing may be used to guide the exhaled air to the environment. For example, the additional length may be 6.3 inches. The additional length of tubing ensures that convective flow dominates over diffusive flow. This prevents backward diffusive flow from distorting sensor readings. The length necessary for prevention is determined using a Péclet Number of 8,000 to 12,000. For example, 10,000. Using a Péclet Number of 10,000 and a length of 6.3 inches provides a safety factor of about 1.58—or 58%. The following equations further explain how this value was determined:

$$Pe = \frac{Lu}{D} \to {L}_{min} = \frac{PeD}{u} = \frac{( 10000 ) {D}_{{CO}_{2} / {O}_{2}}}{1.736 m \text{/} s} \to {L}_{min , {O}_{2}} = 101.4 mm , \\ {L}_{min , {CO}_{2}} = 92.22 mm$$

$${L}_{chosen for tube after sensors} = {L}_{min , {O}_{2}} * ( Safety factor ) \to Safety factor = {L}_{chosen for tube after sensors} / {L}_{min , {O}_{2}}$$

In the previous equations, Pe equals the Péclet Number, Doe equals the diffusion coefficient for O₂ and D_{CO2} equals the diffusion coefficient for CO₂.

Ideally, the device 100 maintains a differential pressure lower than 0.80 cm H₂O, or lower than 10% of positive end-expiratory pressure (PEEP) values, because of the added positive expiratory pressure applied to the lungs when breathing against an additional differential pressure. PEEP occurs when there is an added positive pressure above atmospheric pressure at the end of exhalation. In healthy individuals, the pressure difference at the end of the respiratory cycle (end of exhalation and just before inhalation), is zero. PEEP can occur either intrinsically, due to airway disease, such as COPD, or extrinsically through the use of a mechanical ventilator. Intrinsic PEEP (PEEPi) can occur from incomplete expiration, which causes the alveolar sacs to be partially inflated before inhalation, resulting in hyperinflation and air trapping during the respiratory cycle. Extrinsic PEEP performs the same function, inducing dynamic hyperinflation of the airways to promote increased gas exchange, but it is achieved by adding an additional pressure at the end of exhalation to mechanically ventilated patients. Dynamic hyperinflation of the airways that results in increased gas exchange can mask the effects of lung disease on exhaled concentrations of CO₂ and O₂. The addition of a positive end expiratory pressure can cause this hyperinflation and result in inaccurate CO₂ and O₂ measurements, therefore a low differential pressure from the device is needed to ensure additional dynamic hyperinflation does not occur. Patients with severe lung disease exhibit a PEEPi of approximately 9.8±0.5 cm H₂O. However, extrinsic PEEP only starts to affect hyperinflation and compromise gas exchange at values of 85% or more than intrinsic PEEP. Therefore gas exchange dynamics begin to be affected at PEEP values greater than 7.9 cm H₂O. A threshold of 10% ensures that the added resistance of the device 100 does not create a positive-end expiratory pressure great enough to influence air trapping and gas exchange. Using the exemplary specifications of a radius of 17.5 mm and a length of 266.7 mm, the device 100 maintains a differential pressure of 0.5023 cm H₂O or 6.36% of PEEP values. However, using alternative specifications, the device 100 may maintain a differential pressure between 0.5 cm H₂O to 0.8 cm H₂O or 6%-10% of PEEP values. Additionally, the device 100 may maintain a differential pressure of, for example, less than 7%.

Features and description of device 10 (discussed above) are also applicable to device 100, and are not necessary to repeat herein. The breath analyzer device(s) is/are adapted to provide a differential pressure lower than about 20 percent of positive-end expiratory pressure (less than about 1.6 cm H₂O), preferably a differential pressure lower than about 15 percent of positive-end expiratory pressure (less than about 1.2 cm H₂O), and preferably a differential pressure lower than about 10 percent of positive-end expiratory pressure (less than about 0.8 cm H₂O), such as a differential pressure of, for example, around 7 percent (or thereabouts) of positive-end expiratory pressure.

Therefore, the present invention provides a breath analyzer device having a tube that receives a breath sample without restricting the breathing of the user. The breath analyzer device includes an oxygen sensor to detect the amount of oxygen present in the breath sample and a carbon dioxide sensor to detect the amount of carbon dioxide present in the breath sample. The sensors are adapted to communicate the data to a controller. The breath analyzer device may also include a humidity and temperature sensor to detect the amount of water vapor and temperature of the breath sample respectively. The controller may communicate results determined from the sensors to a display for visual communication to a user. The controller may also communicate results to a wireless or wired interface module which in turn may communicate the results to a remote receiver for further analysis. The breath analyzer device may also include a handle coupled to a mount which may be coupled to the tube. The tube may also have a mouthpiece coupled to the end that receives the breath sample. The mouthpiece may have surfaces coated with a bacteriostatic agent. The mouthpiece may also be disposable or include a filter.

The device of the present invention thus provides a small, hand held device that can determine the levels of oxygen and carbon dioxide in a user's breath when normally exhaling. By evaluating the gas exchange characteristics reflected by the exhaled oxygen and carbon dioxide, the device can obtain information that will be a direct measure of the level of health or disease activity involving these small airways and respiratory components including the alveoli. The present invention thus provides a small, inexpensive, easily applied device that could give information reflecting the ventilation and gas exchange functioning of the lungs, which would be very beneficial in diagnosing and monitoring disease activity in a variety of pulmonary diseases, including asthma, chronic obstructive pulmonary disease (COPD) and others.

The device may be applied at baseline without requiring a change in the user's breathing patterns or having significant flow restriction in the device itself. In this manner can one obtain parameters that truly reflect the state and functioning of the lungs at baseline. There may be times where having the individual go through a respiratory maneuver may be helpful for obtaining specific information, however any respiratory maneuver or restriction to the flow of air will change the characteristics and functioning of the small airways as they function at baseline. For example, if there is restriction to the flow of air as one exhales this will lead to positive end expiratory pressure and keep some of the small airways open artificially and will thus not be a good reflection of the ventilation and gas exchange function of the lungs that the individual experiences normally throughout the day.

There are situations where applying this technology during certain circumstances or with specific respiratory maneuvers might be beneficial for obtaining specific information. An example could include applying this test during exercise as it may be possible to document the dynamic hyperinflation that may occur early in COPD and allow diagnosis at an earlier stage than current technology allows. The ability to obtain these parameters that reflect ventilation and gas exchange while the individual is breathing freely at rest is a beneficial function of this device as this will reflect the state of the lungs at baseline. Other applications of this device may include administering this test during specific situations or during specific maneuvers. Optionally, the device could also be applied to standard spirometers to allow determination of ventilation and gas exchange characteristics with the same device when obtaining standard spirometric parameters.

Changes and modifications in the specifically described embodiments may be carried out without departing from the principles of the present invention, which is intended to be limited only by the scope of the appended claims as interpreted according to the principles of patent law.

1. A breath analyzer device comprising:
a tube for communicating a breath sample from a user of the breath analyzer device, wherein the tube has a first opening wherein the breath sample enters and a second opening where the breath sample exits, and wherein the tube is adapted to provide minimal restrictions on a flow of the breath sample;
an oxygen sensor disposed partially within the tube and adapted to detecting an amount of oxygen present in the breath sample;
a carbon dioxide sensor disposed partially within the tube and adapted to detecting an amount of carbon dioxide present in the breath sample; and
a controller adapted to receive data from the oxygen sensor and the carbon dioxide sensor.

2. The breath analyzer device of claim 1, further comprising a display adapted to receive data from the controller and provide the received data visually.

3. The breath analyzer device of claim 1, further comprising a wireless interface module adapted to receive data from the controller and wirelessly transmit the data to a remote receiver.

4. The breath analyzer device of claim 1, further comprising memory adapted to store data received from the controller.

5. The breath analyzer device of claim 1, further comprising a mouthpiece in fluid communication with the first opening of the tube, wherein the breath sample enters the mouthpiece prior to entering the first opening of the tube.

6. The breath analyzer device of claim 5, wherein the mouthpiece comprises of a surface coated with a bacteriostatic agent or filter to restrict bacteria, viruses or other contaminants from entering the tube.

7. The breath analyzer device of claim 5, further comprising a humidity sensor adapted to detecting an amount of water vapor present in the breath sample, wherein the controller is further adapted to receive data from the humidity sensor.

8. The breath analyzer device of claim 5, further comprising a temperature sensor adapted to detecting a temperature of the breath sample, wherein the controller is further adapted to receive data from the temperature sensor.

9. The breath analyzer device of claim 5, further comprising a handle coupled to the tube for the user to grasp while using the breath analyzer device.

10. The breath analyzer device of claim 1, further comprising a wired interface module adapted to receive data from the controller and transmit the received data through a wired interface to a remote receiver.

11. The breath analyzer device of claim 1, wherein the breath analyzer device wirelessly transmits received data to a remote receiver.

12. The breath analyzer device of claim 1, wherein the breath analyzer device is adapted to provide a differential pressure lower than 0.8 cm H₂O.

13. A breath analyzer device comprising:
a pre-valve subsystem for accepting a breath sample from a user of the breath analyzer device and communicating the breath sample;
at least one check valve for receiving the communicated breath sample and isolating the breath sample from other gases, wherein the at least one check valve is adapted to provide a differential pressure lower than 1.6 cm H₂O;
a carbon dioxide sensor adapted to detecting an amount of carbon dioxide in the breath sample;
an oxygen sensor adapted to detecting an amount of oxygen in the breath sample;
a post-valve subsystem for receiving the communicated breath sample and expelling the breath sample from the breath analyzer device; and
a controller adapted to receive data from the oxygen sensor and the carbon dioxide sensor.

14. The breath analyzer device of claim 13, comprising a heater adapted to heat at least one of the carbon dioxide sensor and oxygen sensor.

15. The breath analyzer device of claim 14, wherein the heater comprises heating tape.

16. The breath analyzer device of claim 13, wherein the pre-valve subsystem has a total dead space of less than 200 ml.

17. The breath analyzer device of claim 13, wherein the pre-valve subsystem has a total dead space of less than 185 ml.

18. The breath analyzer device of claim 13, wherein the post-valve subsystem comprises a post-valve tubing extension adapted to minimize diffusive flow of the breath sample.

19. The breath analyzer device of claim 13, wherein the pre-valve subsystem comprises a disposable mouthpiece.

20. The breath analyzer device of claim 13, wherein the oxygen sensor comprises a Teflon membrane.

21. The breath analyzer device of claim 13, comprising a flow sensor adapted to determine a laminar, transient, or turbulent flow state of the breath sample.

22. The breath analyzer device of claim 13, wherein the breath analyzer device is adapted to provide a differential pressure lower than 0.8 cm H₂O.

23. The breath analyzer device of claim 13, wherein the breath analyzer device is adapted to provide a differential pressure lower than 0.55 cm H₂O.
